(19) United States
(12) Patent Application Publication
(10) Pub. No.: US 2025/0009837 A1
(43) Pub. Date: Jan. 9, 2025

(54) CYCLOSPORINE FORMULATIONS FOR USE IN PATIENTS UNDERGOING CATARACT SURGERY

(71) Applicant: SUN PHARMACEUTICAL INDUSTRIES LIMITED, Mumbai, Maharashtra (IN)

(72) Inventor: John HOVANESIAN, Laguna Beach, CA (US)

(21) Appl. No.: 18/569,563

(22) PCT Filed: Jun. 13, 2022
(86) PCT No.: PCT/IB2022/055462

§ 371 Date: Dec. 12, 2023

Related U.S. Application Data
(60) Provisional application No. 63/202,479, filed on Jun. 14, 2021.

(51) Int. Cl. A61K 38/13; A61K 9/00; A61K 9/107; A61K 47/02; A61K 47/32; A61K 47/44; A61P 27/02

(52) U.S. Cl. CPC A61K 38/13; A61K 9/0048; A61K 9/1075; A61K 47/02; A61K 47/32; A61K 47/44; A61P 27/02

(57) ABSTRACT

A method of preparing patients for cataract surgery includes topical administration of a cyclosporine solution. The administration may occur twice daily and may occur over the course of 28 days. The method results in an improved error prediction for 1-month spherical equivalent refractive outcome and improved ocular surface irregularity.

### BACKGROUND OF THE INVENTION

Patient satisfaction after cataract surgery in today's world relies upon minimizing the refractive error and optimizing the quality of the visual image (see D1, D2). Ocular surface irregularity adversely impacts the consistency of corneal measurements preoperatively and can continue to affect visual quality post-surgery (see D3-D5). These inconsistent preoperative corneal measurements directly impact the ability of cataract surgeons to choose the proper intraocular lens (IOL) implant. It follows that improving the ocular surface, for example in patients with dry eye disease (DED), preoperatively will lead to greater refractive accuracy in lens selection (see D6-D8).

It is well established that the management of ocular surface disease is critical to the success of cataract surgery for several reasons. Not only can cataract surgery exacerbate dry eye signs and symptoms, even in patients who self-report as asymptomatic, but ocular surface irregularities can compromise the accuracy of presurgical biometry and corneal topography/tomography, causing inaccurate IOL calculations, suboptimal postoperative outcomes, and patient dissatisfaction (see D9, D10). Higher Order Aberrations (HOAs) have also been linked to postoperative patient dissatisfaction, especially if multifocal IOLs were implanted. Given the generally high patient postoperative expectations (see D11), cataract surgeons must prioritize minimizing these issues to meet patient needs.

### DOCUMENT REFERENCES

D1. Monestam E. Long-term outcomes of cataract surgery: 15-year results of a prospective study. J Cataract Refract Surg. January 2016; 42 (1): 19-26. doi:10.1016/j.jcrs.2015.07.040
D2. Gollogly H E, Hodge D O, St Sauver J L, Erie J C. Increasing incidence of cataract surgery: population-based study. J Cataract Refract Surg. September 2013; 39 (9): 1383-9. doi:10.1016/j.jcrs.2013.03.027
D3. Lee A C, Qazi M A, Pepose J S. Biometry and intraocular lens power calculation. Current opinion in ophthalmology. January 2008; 19 (1): 13-7. doi:10.1097/ICU.0b013e3282flc5ad
D4. Jeong J, Song H, Lee J K, Chuck R S, Kwon J W. The effect of ocular biometric factors on the accuracy of various IOL power calculation formulas. BMC Ophthalmol. May 2 2017; 17 (1): 62. doi:10.1186/s12886-017-0454-y
D5. Norrby S. Sources of error in intraocular lens power calculation. J Cataract Refract Surg. March 2008; 34 (3): 368-76. doi:10.1016/j.jcrs.2007.10.031
D6. Sheard R. Optimising biometry for best outcomes in cataract surgery. Eye. February 2014; 28 (2): 118-25. doi:10.1038/eye.2013.248
D7. Sahin A, Hamrah P. Clinically relevant biometry. Current opinion in ophthalmology. January 2012; 23 (1): 47-53. doi:10.1097/ICU.0b013e32834cd63e
D8. Hovanesian J, Epitropoulos A, Donnenfeld E D, Holladay J T. The Effect of Lifitegrast on Refractive Accuracy and Symptoms in Dry Eye Patients Undergoing Cataract Surgery. Clin Ophthalmol. 2020; 14:2709-2716. doi:10.2147/opth.S264520
D9. Li X M, Hu L, Hu J, Wang W. Investigation of dry eye disease and analysis of the pathogenic factors in patients after cataract surgery. Cornea. October 2007; 26 (9 Suppl 1): S16-20. doi:10.1097/ICO.0b013e31812f67ca00003226-200710001-00005 [pii]
D10. Epitropoulos A T, Matossian C, Berdy G J, Malhotra R P, Potvin R. Effect of tear osmolarity on repeatability of keratometry for cataract surgery planning. J Cataract Refract Surg. August 2015; 41 (8): 1672-7. doi:10.1016/j.jcrs.2015.01.016
D11. Addisu Z, Solomon B. Patients' preoperative expectation and outcome of cataract surgery at jimma university specialized hospital-department of ophthalmology. Ethiop J Health Sci. March 2011; 21 (1): 47-55. doi:10.4314/ejhs.v21i1.69044

### SUMMARY OF THE INVENTION

The present inventions relates to a method of preparing a subject for cataract surgery, the method comprising: administering a solution comprising cyclosporine to an eye on which cataract surgery is to be performed.

In one embodiment of the instant method, the cyclosporine solution is topically administered.

In another embodiment of the instant invention, the cyclosporine solution is administered twice daily.

In yet another embodiment of the instant invention, the cyclosporine solution is administered for 28 days immediately prior to the cataract surgery.

In still yet another embodiment of the instant invention, the solution comprising cyclosporine is an ophthalmic aqueous topical formulation consisting of about 0.087-0.093 wt % cyclosporine, about 1.0 wt % hydrogenated 40 polyoxyl castor oil, about 0.05 wt % octoxynol-40, about 0.3 wt % povidone, about 0.05 wt % sodium chloride, about 0.20-0.405 wt % sodium phosphate monobasic and about 0.23-0.465 wt % sodium phosphate dibasic, adjusted to a pH of about 5 to about 8 with sodium hydroxide/hydrochloric acid, and wherein the final volume is made up with water. In an aspect of the above embodiment the sodium phosphate monobasic about 0.20-0.405 wt % is equivalent to sodium phosphate monobasic dihydrate about 0.26-0.53 wt %. In another aspect of the above embodiment the sodium phosphate dibasic is in an anhydrous form.

In one embodiment of the instant invention, the cyclosporine is present in an amount of about 0.09 wt % of the formulation.

In another embodiment of the present invention, the pH of the solution of cyclosporine is about 6.6 to 7.0.

In yet another embodiment of the present invention, the solution comprising cyclosporine is an aqueous clear nanomicellar ophthalmic formulation comprising about 0.05-0.5% by weight cyclosporine, a polyalkoxylated alcohol and one or more polymers comprising HCO-40, HCO-60, HCO-80, HCO-100, polyoxyl 35 castor oil or combinations thereof.

In still yet another embodiment of the present invention, the one or more polymers comprise HCO-40, HCO-60, HCO-80, HCO-100 or combinations thereof.

In one embodiment of the present invention, said polymer comprises polyoxyl 35 castor oil.

In another embodiment of the present invention, the polymer is HCO-40.

In one embodiment of the present invention, the polymer is about 0.5-1.5% by weight of the cyclosporine formulation.

In another embodiment of the present invention, the polymer comprises HCO-40 HCO-60, HCO-80, or combinations thereof and is about 0.5-1.5% by weight of the formulation.

In yet another embodiment of the present invention, the polyalkoxylated alcohol used in the solution of cyclosporine comprises Octoxynol-40.

In still yet another embodiment of the present invention, the polyalkoxylated alcohol comprises Octoxynol-40 and is about 0.02-4% by weight of the formulation.

In another embodiment of the present invention, the polyalkoxylated alcohol comprises Octoxynol-40 and is about 0.02-0.1% by weight of the formulation.

In another embodiment of the present invention, the method uses a solution of cyclosporine, wherein the cyclosporine is about 0.05-0.2% by weight of the formulation.

In yet another embodiment of the instant method, the polymer used in the solution of cyclosporine comprises HCO-40, HCO-60, HCO-80, HCO-100 or combinations thereof; and wherein said polyalkoxylated alcohol is Octoxynol-40.

In yet another embodiment of the instant method, the polymer used in the solution of cyclosporine is about 0.5-1.5% by weight of the formulation; and said polyalkoxylated alcohol is Octoxynol-40 and is about 0.02-0.1% by weight of the formulation.

In yet another embodiment of the instant method, the polymer used in the solution of cyclosporine is about 0.5-1.5% by weight of the formulation; said polyalkoxylated alcohol comprises Octoxynol-40 in an amount of about 0.02-0.1% by weight of the formulation; and the cyclosporine is about 0.05-0.2% by weight of the formulation.

Additionally, the present invention provides a method of reducing ocular surface irregularity, the method comprising: administering a solution comprising cyclosporine to an eye.

In one embodiment of the present invention, the cyclosporine solution is topically administered.

In another embodiment, the cyclosporine solution is administered twice daily.

In yet another embodiment, the cyclosporine solution is administered for 28 days.

In still yet another embodiment, the solution comprising cyclosporine is an ophthalmic aqueous topical formulation consisting of about 0.087-0.093 wt % cyclosporine, about 1.0 wt % hydrogenated 40 polyoxyl castor oil, about 0.05 wt % octoxynol-40, about 0.3 wt % povidone, about 0.05 wt % sodium chloride, about 0.20-0.405 wt % sodium phosphate monobasic and about 0.23-0.465 wt % sodium phosphate dibasic, adjusted to a pH of about 5 to about 8 with sodium hydroxide/hydrochloric acid, and wherein the final volume is made up with water.

In one embodiment of the present invention, the cyclosporine is present in an amount of about 0.09 wt % of the formulation.

In another embodiment of the present invention, the pH of the solution of cyclosporine is about 6.6 to 7.0.

In yet another embodiment of the present invention, the method of reducing ocular surface irregularity administers a cyclosporine solution comprising an aqueous clear nanomicellar ophthalmic formulation comprising about 0.05-0.5% by weight cyclosporine, a polyalkoxylated alcohol and one or more polymers comprising HCO-40, HCO-60, HCO-80, HCO-100, polyoxyl 35 castor oil or combinations thereof.

In yet another embodiment of the present invention, the polymer comprises HCO-40, HCO-60, HCO-80, HCO-100 or combinations thereof.

In yet another embodiment of the present invention, the polymer comprises polyoxyl 35 castor oil.

In yet another embodiment of the present invention, the polymer is HCO-40.

In still yet another embodiment of the present invention, the polymer is about 0.5-1.5% by weight of the formulation.

In an embodiment of the present invention, the method of reducing ocular surface irregularity administers a cyclosporine solution comprising an aqueous clear nanomicellar ophthalmic formulation comprising cyclosporine, a polyalkoxylated alcohol and one or more polymers, wherein the polymer comprises HCO-40 HCO-60, HCO-80, or combinations thereof and is about 0.5-1.5% by weight of the formulation.

In another embodiment of the present invention, the method of reducing ocular surface irregularity administers a cyclosporine solution comprising an aqueous clear nanomicellar ophthalmic formulation comprising cyclosporine, a polyalkoxylated alcohol and one or more polymers, wherein said polyalkoxylated alcohol comprises Octoxynol-40.

In yet another embodiment, the method of reducing ocular surface irregularity administers a cyclosporine solution comprising an aqueous clear nanomicellar ophthalmic formulation comprising cyclosporine, a polyalkoxylated alcohol and one or more polymers, wherein the polyalkoxylated alcohol comprises Octoxynol-40 and is 0.02-4% by weight of the formulation.

In still yet another embodiment of the present invention, the polyalkoxylated alcohol comprises Octoxynol-40 and is 0.02-0.1% by weight of the formulation.

In an embodiment of the present invention, the method of reducing ocular surface irregularity uses a solution of cyclosporine, wherein the cyclosporine is 0.05-0.2% by weight of the formulation.

In another embodiment of the present invention, the method of reducing ocular surface irregularity uses a solution of cyclosporine solution, wherein said solution comprises a polymer comprises HCO-40, HCO-60, HCO-80, HCO-100 or combinations thereof; and wherein said polyalkoxylated alcohol is Octoxynol-40.

In yet another embodiment of the present invention, said polymer is about 0.5-1.5% by weight of the formulation; and said polyalkoxylated alcohol is Octoxynol-40 and is about 0.02-0.1% by weight of the formulation.

In still yet another embodiment of the present invention, said polymer is about 0.5-1.5% by weight of the formulation; said polyalkoxylated alcohol comprises Octoxynol-40 and is about 0.02-0.1% by weight of the formulation; and the cyclosporine is 0.05-0.2% of the formulation.

The instant invention also provides a method of reducing conjunctival erythema, the method comprising: administering a solution comprising cyclosporine to an eye.

In one embodiment of the present invention, the cyclosporine solution is topically administered.

In another embodiment of the present invention, the cyclosporine solution is administered twice daily.

In one embodiment of the present invention, the cyclosporine solution is administered for 28 days.

In one embodiment, the present invention provides a method of reducing conjunctival erythema, the method comprising administering a solution comprising cyclosporine to an eye, wherein the solution comprising cyclosporine is an ophthalmic aqueous topical formulation consisting of about 0.087-0.093 wt % cyclosporine, about 1.0 wt % hydrogenated 40 polyoxyl castor oil, about 0.05 wt % octoxynol-40, about 0.3 wt % povidone, about 0.05 wt % sodium chloride, about 0.20-0.405 wt % sodium phosphate monobasic and about 0.23-0.465 wt % sodium phosphate dibasic, adjusted to a pH of about 5 to about 8 with sodium hydroxide/hydrochloric acid, and wherein the final volume is made up with water.

In another embodiment of the present invention, the cyclosporine is present in an amount of about 0.09 wt % of the formulation.

In yet another embodiment of the present invention, the pH of the formulation is about 6.6 to 7.0.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising about 0.05-0.5% by weight cyclosporine, a polyalkoxylated alcohol and one or more polymers comprising HCO-40, HCO-60, HCO-80, HCO-100, polyoxyl 35 castor oil or combinations thereof.

In one embodiment of the present invention, the polymer comprises HCO-40, HCO-60, HCO-80, HCO-100 or combinations thereof.

In another embodiment of the present invention, the polymer comprises polyoxyl 35 castor oil.

In still yet another embodiment, the polymer is HCO-40.

In one embodiment of the present invention, the polymer is about 0.5-1.5% by weight of the formulation.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein the Substitute Specification (clean) filed: Aug. 13, 2024 polymer comprises HCO-40 HCO-60, HCO-80, or combinations thereof and is about 0.5-1.5% by weight of the formulation.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein said polyalkoxylated alcohol comprises Octoxynol-40.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein the polyalkoxylated alcohol comprises Octoxynol-40 and is about 0.02-4% by weight of the formulation.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein the polyalkoxylated alcohol comprises Octoxynol-40 and is about 0.02-0.1% by weight of the formulation.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein the cyclosporine is about 0.05-0.2% by weight of the formulation.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein said polymer comprises HCO-40, HCO-60, HCO-80, HCO-100 or combinations thereof; and wherein said polyalkoxylated alcohol is Octoxynol-40.

In another embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein said polymer is about 0.5-1.5% by weight of the formulation; and said polyalkoxylated alcohol is Octoxynol-40 and is about 0.02-0.1% by weight of the formulation.

In one embodiment, the present invention provides a method of reducing conjunctival erythema comprising administering a solution comprising cyclosporine to an eye, wherein the solution comprising cyclosporine is an aqueous clear nanomicellar ophthalmic formulation comprising cyclosporine, a polyalkoxylated alcohol and one or more polymers, wherein said polymer is about 0.5-1.5% by weight of the formulation; said polyalkoxylated alcohol comprises Octoxynol-40 and is about 0.02-0.1% by weight of the formulation; and the cyclosporine is about 0.05-0.2% by weight of the formulation.

### BRIEF DESCRIPTION OF THE SEVERAL VIEWS OF THE DRAWINGS

FIG. 1 is a graphical representation illustrating the process of study visits and examinations.

FIG. 2 is a graph showing the predictive accuracy of corneal power measurements performed after cyclosporine was significantly higher than when performed before cyclosporine (n=64, P<0.03, paired t-test).

FIG. 3 is a graph showing that significantly more patients had improvement than a decline in root-mean-square higher order aberrations after 28 days of cyclosporine (n=64, P<0.0001, McNemar's Chi-squared test).

FIG. 4 is a graph showing that SPEED scores improved significantly after 28 days of treatment with cyclosporine (P<0.00001, paired t-test).

FIG. 5 is a graph showing that Corneal staining (Oxford Scale) was measured before and after 28 days of treatment with cyclosporine and improved significantly following treatment, with complete resolution in 56% of patients (P<0.000001, paired t-test). Note all eyes (n=64) had at least grade 1 staining before treatment as a condition of enrolling in the study.

FIG. 6 is a graph showing tear breakup time, which was measured before and after 28 days of treatment with cyclosporine, and improved significantly following treatment (n=64, P<0.00001, paired t-test).

### DETAILED DESCRIPTION OF THE INVENTION

The present disclosure relates to the topical administration of a solution containing cyclosporine, for example cyclosporine about 0.09% (CEQUA), in patients presenting for cataract surgery for 28 days pre-surgery, and illustrates an improvement in surface regularity and the predictive accuracy of preoperative corneal power measurements. This present disclosure also describes the impact of topical cyclosporine solutions on irregularity of the ocular surface as measured by HOAs, corneal staining, tear breakup time (TBUT), and ocular redness.

As used herein, the term “polyoxyl lipid or fatty acid” refers to mono- and diesters of lipids or fatty acids and polyoxyethylene diols. Polyoxyl lipids or fatty acids may be numbered (“n”) according to the average polymer length of the oxyethylene units (e.g., 40, 60, 80, 100) as is well understood in the art. The term “n−40 polyoxyl lipid” means that the ployoxyl lipid or fatty acid has an average oxyethylene polymer length equal to or greater than 40 units. Stearate hydrogenated castor oil and castor oil are common lipids/fatty acids commercially available as polyoxyl lipids or fatty acid, however, it is understood that any lipid or fatty acid could be polyoxylated to become a polyoxyl lipid or fatty acid as contemplated herein. Examples of polyoxyl lipid or fatty acids include without limitation HCO-40, HCO-60, HCO-80, HCO-100, polyoxyl 40 stearate, polyoxyl 35 castor oil.

As used herein, the term sodium phosphate monobasic and/or sodium phosphate dibasic, is used to denote phosphate buffer, and can also be used in different hydrate and anhydrous forms of the salts. For instance, sodium phosphate monobasic can be used interchangeably to equivalent molar amounts of sodium phosphate monobasic dihydrate, and sodium phosphate dibasic can be used interchangeably to equivalent molar amounts of sodium phosphate dibasic anhydrous.

In some embodiments of any of the compositions and methods described herein, the average polymer length of the oxyethylene units of a polyoxyl lipid or fatty acid is longer for a relatively larger active ingredient and is shorter for a relatively smaller active ingredient; for example in some embodiments in which the active ingredient is a resolvin or resolvin-like compound the polyoxyl lipid is HCO-60 and in some embodiments where the active ingredient is cyclosporine A (which is larger than a resolvin) the polyoxyl lipid is HCO-80 or HCO-100.

As used herein, the term “micelle” or “nanomicelle” refers to an aggregate (or cluster) of surfactant molecules. Micelles only form when the concentration of surfactant is greater than the critical micelle concentration (CMC). Surfactants are chemicals that are amphipathic, which means that they contain both hydrophobic and hydrophilic groups. Micelles can exist in different shapes, including spherical, cylindrical, and discoidal. A micelle comprising at least two different molecular species is a mixed micelle. In some embodiments, ophthalmic compositions of the present disclosure include an aqueous, clear, mixed micellar solution.

As used throughout the specification the term “about” is considered to include ±10% of the numerical value of the quantities used in the formulation/composition according to the present invention.

A patient or subject to be treated by any of the compositions or methods of the present disclosure can mean either a human or a non-human animal. In an embodiment, the present disclosure provides methods for the treatment of preparing a human subject for cataract surgery. In an embodiment, the present disclosure provides methods for reducing ocular surface irregularity in a human patient in need thereof. In an embodiment, the present disclosure provides methods for reducing conjunctival erythema in a human patient in need thereof. In another embodiment, the present disclosure provides for the methods above for a veterinary patient in need thereof, including, but not limited to dogs, horses, cats, rabbits, gerbils, hamsters, rodents, birds, aquatic mammals, cattle, pigs, camelids, and other zoological animals.

### Cyclosporine 0.09% Solution

A cyclosporine solution useful according to the present disclosure is a solution comprising cyclosporine as disclosed in, for example, U.S. Pat. Nos. 8,980,839; 9,937,225; 10,441,630; and 10,918,694; the entire contents of each of which are incorporated by reference in their entirety.

In preferred embodiments, the solution comprising cyclosporine is as disclosed in U.S. Pat. No. 10,918,694 and consists of an ophthalmic aqueous topical formulation consisting of about 0.087-0.093 wt % cyclosporine, about 1.0 wt % hydrogenated 40 polyoxyl castor oil, about 0.05 wt % octoxynol-40, about 0.3 wt % povidone, about 0.05 wt % sodium chloride, about 0.20-0.405 wt % sodium phosphate monobasic, and about 0.23-0.465 wt % sodium phosphate dibasic, adjusted to a pH of about 5 to about 8 with sodium hydroxide/hydrochloric acid, and wherein the final volume is made up with water. In certain arrangements of this embodiment, the cyclosporine is present in an amount of about 0.09 wt % of the formulation. In other arrangements of this embodiment, the pH of the formulation is about 6.6 to 7.0.

In certain preferred embodiments, the solution comprising cyclosporine is CEQUA.

In another embodiment, the solution comprising cyclosporine may be as disclosed in U.S. Pat. No. 9,937,225. In such an embodiment, the solution comprising cyclosporine may be an aqueous clear nanomicellar ophthalmic formulation comprising about 0.05-0.5% by weight cyclosporine, a polyalkoxylated alcohol and one or more polymers comprising HCO-40, HCO-60, HCO-80, HCO-100, polyoxyl 35 castor oil or combinations thereof. In certain arrangements of this embodiment, the polymer may comprise HCO-40, HCO-60, HCO-80, HCO-100 or combinations thereof. In other arrangements of this embodiment, the polymer may comprise polyoxyl 35 castor oil. In still other arrangements of this embodiment, the polymer is HCO-40. In certain arrangements of this embodiment, the polymer may be present in an amount of about 0.5-1.5% by weight of the formulation. In certain arrangements of this embodiment, the polymer may comprise HCO-40 HCO-60, HCO-80, or combinations thereof and may be present in an amount of about 0.5-1.5% by weight of the formulation. In still other arrangements of this embodiment, the polyalkoxylated alcohol may comprise Octoxynol-40, and in arrangements in which the polyalkoxylated alcohol comprises Octoxynol-40, the Octoxynol-40 may be present in an amount of about 0.02-4% by weight or about 0.02-0.1% by weight of the formulation. In certain arrangements of this embodiment, the cyclosporine may be present in an amount of about 0.05-0.2% by weight of the formulation. In other arrangements of this embodiment, the polymer may comprise HCO-40, HCO-60, HCO-80, HCO-100 or combinations thereof; and the polyalkoxylated alcohol may be Octoxynol-40. In still other arrangements of this embodiment, the polymer may be present in an amount of about 0.5-1.5% by weight of the formulation; and the polyalkoxylated alcohol may be Octoxynol-40 in an amount of about 0.02-0.1% by weight of the formulation. In another arrangement of this embodiment, the polymer may be present in an amount of about 0.5-1.5% by weight of the formulation, the polyalkoxylated alcohol may comprise Octoxynol-40 in an amount of about 0.02-0.1% of the formulation, and the cyclosporine may be present in an amount of about 0.05-0.2% of the formulation.

### Additional Formulation Ingredients

The compositions of the present disclosure may also contain other components such as, but not limited to, additives, adjuvants, buffers, tonicity agents, bioadhesive polymers, and preservatives. In any of the compositions of this disclosure for topical to the eye, the mixtures are preferably formulated at about pH 5 to about pH 8. This pH range may be achieved by the addition of buffers to the composition as described in the examples. In an embodiment, the pH range in the composition in a formulation is about pH 6.4 to about 7.5. In yet another embodiment, the pH range in the composition in a formulation is about pH 6.6 to about pH 7.0. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values. The mixed micellar compositions of the present disclosure are stable in buffered aqueous solution. That is, there is no adverse interaction between the buffer and any other component that would cause the compositions to be unstable.

Tonicity agents include, for example, mannitol, sodium chloride, xylitol, etc. These tonicity agents may be used to adjust the osmolality of the compositions. In one aspect, the osmolality of the formulation is adjusted to be in the range of about 250 to about 350 mOsmol/kg. In a preferred aspect, the osmolality of the formulation is adjusted to between about 280 to about 300 mOsmol/kg.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the calcineurin inhibitor or mTOR inhibitor, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose. In an embodiment, an aqueous, clear, mixed micellar solution of the present disclosure includes additives such as sugars.

In an embodiment, compositions of the present disclosure further comprise one or more bioadhesive polymers. Bioadhesion refers to the ability of certain synthetic and biological macromolecules and hydrocolloids to adhere to biological tissues. Bioadhesion is a complex phenomenon, depending in part upon the properties of polymers, biological tissue, and the surrounding environment. Several factors have been found to contribute to a polymer's bioadhesive capacity: the presence of functional groups able to form hydrogen bridges (—OH, COOH), the presence and strength of anionic charges, sufficient elasticity for the polymeric chains to interpenetrate the mucous layer, and high molecular weight. Bioadhesion systems have been used in dentistry, orthopedics, ophthalmology, and in surgical applications. However, there has recently emerged significant interest in the use of bioadhesive materials in other areas such as soft tissue-based artificial replacements, and controlled release systems for local release of bioactive agents. Such applications include systems for release of drugs in the buccal or nasal cavity, and for intestinal or rectal administration.

In an embodiment, a composition of the present disclosure includes at least one bioadhesive polymer. The bioadhesive polymer can enhance the viscosity of the composition and thereby increase residence time in the eye. Bioadhesive polymers of the present disclosure include, for example, carboxylic polymers like CARBOPOL (carbomers), NOVEON. (polycarbophils), cellulose derivatives including alkyl and hydroxyalkyl cellulose like methylcellulose, hydroxypropylcellulose, carboxymethylcellulose, gums like locust beam, xanthan, agarose, karaya, guar, and other polymers including but not limited to polyvinyl alcohol, polyvinyl pyrollidone, polyethylene glycol, PLURONIC (Poloxamers), tragacanth, and hyaluronic acid; phase-transition polymers for providing sustained and controlled delivery of enclosed medicaments to the eye (e.g., alginic acid, carrageenans (e.g., Eucheuma), xanthan and locust bean gum mixtures, pectins, cellulose acetate phthalate, alkylhydroxyalkyl cellulose and derivatives thereof, hydroxyalkylated polyacrylic acids and derivatives thereof, poloxamers and their derivatives, etc. Physical characteristics in these polymers can be mediated by changes in environmental factors such as ionic strength, pH, or temperature alone or in combination with other factors. In an embodiment, the optional one or more bioadhesive polymers is present in the composition from about 0.01 wt % to about 10 wt %/volume, preferably from about 0.1 to about 5 wt %/volume. In an embodiment, the compositions of the present disclosure further comprise at least one hydrophilic polymer excipient selected from, for example, PVP-K-30, PVP-K-90, HPMC, HEC, and polycarbophil. In an embodiment, the polymer excipient is selected from PVP-K-90, PVP-K-30 or HPMC. In an embodiment, the polymer excipient is selected from PVP-K-90 or PVP-K-30.

In an embodiment, if a preservative is desired, the compositions may optionally be preserved with any of many well-known preservatives, including benzyl alcohol with/without EDTA, benzalkonium chloride, chlorhexidine, COSMOCIL CQ, or DOWICIL 200. In certain embodiments, it may be desirable for a formulation as described herein to not include any preservatives. In this regard, preservatives may in some embodiments not be necessary or desirable in formulations included in single use containers. In other embodiments, it may be advantageous to include preservatives, such as in certain embodiments in which the formulations are included in a multiuse container.

In some embodiments of the compositions and methods disclosed herein, the cyclosporine further comprises one or more additional active ingredients, e.g., active agents selected from the group consisting of a resolvin or resolvin-like compound, a steroid (such as a corticosteroid), and the like. In some embodiments, the additional active agent includes a resolvin. In some embodiments, the additional active agent includes a corticosteroid. In some embodiments, the additional active agent includes a resolvin and a corticosteroid. In some embodiments, the additional active agent includes an antibiotic, for example one or more antibiotics selected from the group consisting of azythromycin, ciprofloxacin, ofloxacin, gatifloxacin, levofloxacin, moxifloxacin, besifloxacin, and levofloxacin. In some embodiments, the additional active agent includes an antibiotic, for example one or more antibiotics selected from the group consisting of azythromycin, ciprofloxacin, ofloxacin, gatifloxacin, levofloxacin, moxifloxacin, besifloxacin, and levofloxacin; and a second of such agents is a resolvin. In some embodiments, the active agent includes two or more active agents and one of said active agents is an antiviral, for example one or more antivirals selected from the group consisting of ganciclovir, trifluridine, acyclovir, famciclovir, valacyclovir, penciclovir and cidofovir. In some embodiments, the active agent includes two or more active agents and one of the active agents is an antibiotic, for example one or more antivirals selected from the group consisting of ganciclovir, trifluridine, acyclovir, famciclovir, valacyclovir, penciclovir and cidofovir; and a second of the active agents is a resolvin.

### Method of Administration

The solution comprising cyclosporine is preferably administered topically to an eye. The method of administration may be as described in, for example, U.S. Pat. Nos. 8,980,839; 9,937,225; 10,441,630; and 10,918,694.

In the Experiments reported below, a cyclosporine 0.09% solution (CEQUA) was administered twice daily for 28 days to patients. However, the method of administration is not limited to the method shown. Administration may occur for as long or as short a period as necessary to improve ocular surface irregularity. Doses may also be altered as may be necessary to achieve improvement in ocular surface irregularity.

### Experiments

In the Experiments reported below, a cyclosporine 0.09% solution (CEQUA) was used.

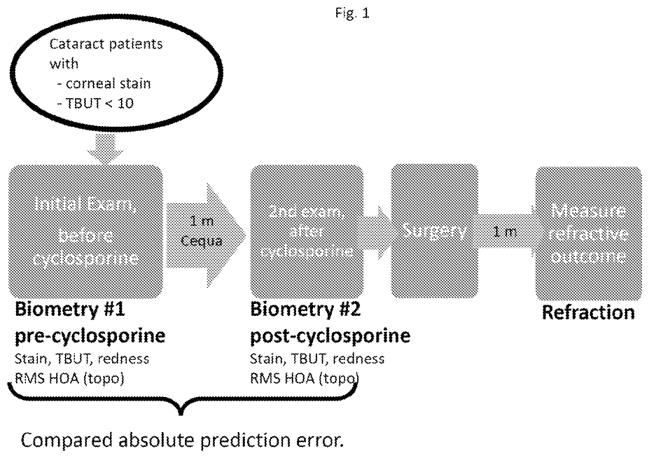

FIG. 1 generally shows the procedures used in testing patients. The following experiments involved an open-label, multicenter, prospective study that included 75 eyes (from 75 patients) who had presented for cataract surgery evaluation with signs of DED, including corneal staining with fluorescein and a TBUT of ≤10 seconds. Each patient underwent the same set of presurgical diagnostics before and after treatment with cyclosporine 0.09%. Patients who were scheduled to have both eyes undergo cataract surgery had their first eye enrolled in the study.

The inclusion/exclusion criteria and study protocol were previously published in D8. Exclusion criteria included previous ocular surgery within the past 3 months, ocular inflammation or corneal scarring, corneal dystrophy, or other defect or abnormality of the ocular surface. Subjects were permitted to continue unchanged any baseline dry eye treatments (lubricant drops, warm compresses) but not make any modifications beyond the addition of topical cyclosporine 0.09%.

At the 1-month visit following cyclosporine 0.09% treatment, patients were asked to report on their compliance, and noncompliant patients were excluded.

The patients were prescribed topical cyclosporine 0.09% for 28 days BID. As discussed below, corneal curvature measurements, slit lamp exam, and Standardized Patient Evaluation of Eye Dryness (SPEED) questionnaire were evaluated at the initial and follow-up visits. Cataract surgery occurred 1 to 3 weeks after the second biometry visit. Refraction and corrected distance visual acuity measurements were performed 1-month post-surgery. The primary outcome was the difference in absolute prediction error of 1-month spherical equivalent refractive outcome before and after cyclosporine treatment. Secondary outcomes included the effect of topical cyclosporine 0.09% on ocular surface irregularity.

Corneal curvature measurements were performed at both the initial visit and after 28 days of cyclosporine 0.09% using an IOL Master 500 or 700 (CARL ZEISS MEDITEC, California, US). Corneal topography was collected with a ZEISS ATLAS 900 or later topographer, and root-mean-square (RMS) HOAs were recorded in the central 6.0 mm of the cornea for each visit. Other assessments included slit lamp examination (conjunctival hyperemia by the Schulze scale (see D3 and D5), corneal staining by the Oxford grading scale, and TBUT). The Standardized Patient Evaluation of Eye Dryness (SPEED) survey was also administered, and scores >10 were considered abnormal. See D3, D6. Following the initial evaluation, patients were prescribed 28 days of cyclosporine 0.09% BID. At the end of the 28-day cycle, biometry and exam measurements were repeated. Cataract surgery was performed 1 to 3 weeks after the second evaluation using the intraocular lens (IOL) power suggested by the biometry measurements from the latter evaluation.

Approximately 1 month postoperatively, a refraction and corrected distance visual acuity (CDVA) measurement was performed.

Each set of the biometry measurements was used to generate an IOL power calculation for the study eye using the Barrett Universal II Formula; the predicted manifest refraction spherical equivalent of the IOL selected for surgery was calculated for each set of measurements. These predicted spherical equivalents were then compared against the actual final manifest refraction spherical equivalent measured 30 days after surgery to determine each prediction's absolute measurement error.

SPEED questionnaire scores, conjunctival hyperemia scores, corneal staining, and TBUT were recorded for each visit and entered into a database for comparison before and after surgery. Paired t-testing was used to evaluate the difference for statistical significance with 95% confidence. Paired t-tests were also used to compare RMS HOAs measurements before and after cyclosporine 0.09% treatment.

A total of 75 patients were initially enrolled. Of these, 8 (11%) withdrew because of a renewed SARS-COV-2-related stay-at-home order by cancelling their surgery or withdrawing from study participation to reduce clinic visits. Two (3%) cancelled due to insurance or scheduling conflicts, and 1 (1%) withdrew because the study medication was not tolerable. Of the 64 patients who completed the study, 34 (53%) were female and 30 (47%) were male. A total of 36 patients (56%) had their right eyes studied and 28 (44%) had their left. The mean age was 70.5+7.4 years (range 50 to 84). Patients were enrolled from Harvard Eye Associates (n=27), Ophthalmology Associates (n=20), and Ophthalmic Surgeons and Consultants of Ohio (n=17).

### Measurement of Primary Outcome

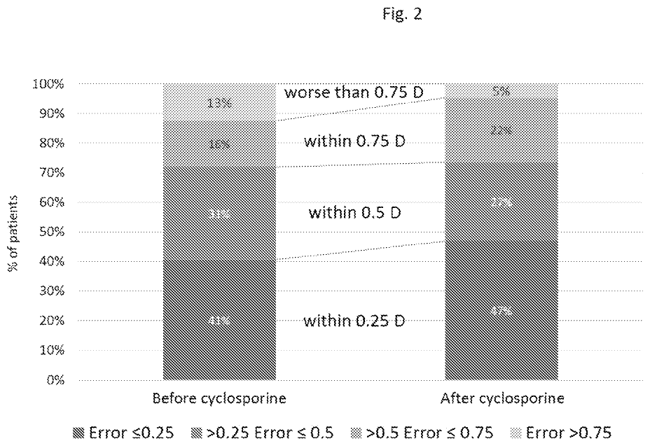

FIG. 2 is a graph showing the predictive accuracy of corneal power measurements performed before and after cyclosporine treatments. The absolute prediction error of 1-month spherical equivalent refractive outcome was 0.39±0.30 D vs 0.33±0.25 D based on biometry performed before and after treatment with cyclosporine 0.09%, respectively. This difference was statistically significant (P<0.03, paired t-test). The proportion of eyes that would have achieved the target refraction was greater after cyclosporine 0.09%: 41% vs 47% within 0.25 D (P<05, McNemar's Chi-squared test), 72% vs 73% within 0.5 D (P<31), and 88% vs 95% within 0.75 D (P<0.03). These differences were statistically significant for accuracy within 0.25 D and 0.75 D.

### Measurement of Secondary Outcomes

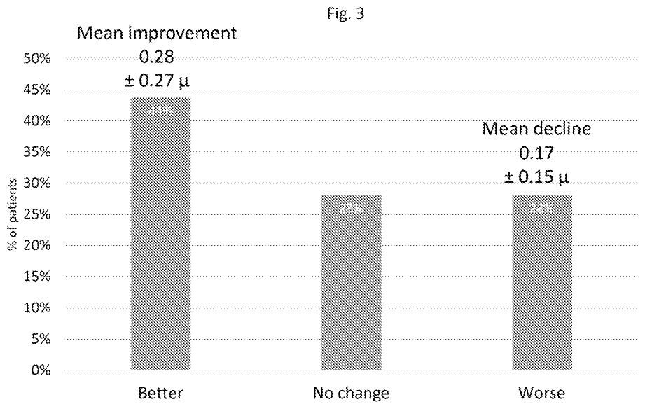

FIG. 3 shows the results of the measurements of RMS HOA, and shows that more patients had improvement versus a decline in RMS HOA after 28 days of treatment with cyclosporine. As illustrated in FIG. 3, the cyclosporine 0.09% treatment caused changes in total HOAs measured within the central 6.0 mm of the cornea, with improvement by a mean of 0.28+0.27μ in 28 (44%) of eyes, no change in 18 (28%), and worsening by a mean of 0.17±0.15μ in 18 (28%) of eyes. These differences were statistically significant, favoring improvement (P<0. 0001, McNemar's Chi-squared test). The overall mean magnitude of corneal HOAs was also significantly improved by cyclosporine 0.09% with a value of 0.68±0.32μ before treatment and 0.60±0.22μ after (P<0.02, paired t-test).

The presence of total corneal HOAs greater than 0.5μ before surgery are associated with suboptimal subjective patient perceptions postoperatively. Using this cutoff as a measure of probability for success with a multifocal IOL, 25 (39%) patients before and 29 (45%) patients after cyclosporine 0.09% treatment would be considered candidates for a multifocal IOL. This difference was statistically significant (P<0.05, McNemar's Chi-squared test).

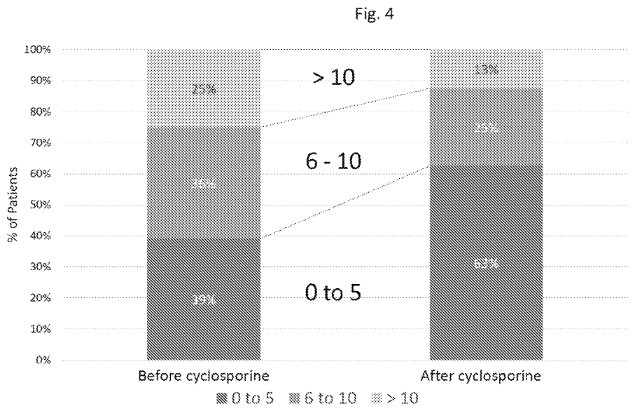

FIG. 4 shows the result of the SPEED scores. As can be seen in FIG. 4, SPEED scores improved significantly after 28 days of treatment with 0.09% cyclosporine, with a mean score of 7.9±6.2 before and 5.2±5.3 after (P<0.00001, paired t-test). Scores of ≤5 were observed in 25 (39%) patients before and 40 (63%) patients after cyclosporine 0.09% treatment, and scores <10 were noted in 48 (75%) patients before and 56 (88%) patients after cyclosporine 0.09% treatment. These differences were statistically significant (P<0.007 and P<0.04, respectively, McNemar's Chi-squared test).

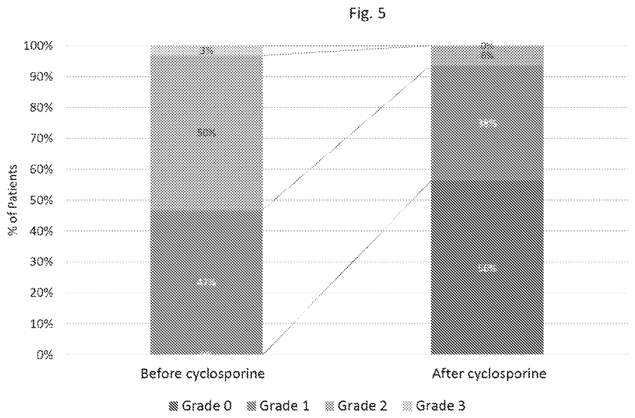

FIG. 5 shows the results of corneal staining (Oxford Scale) testing, measured before and after 28 days of treatment with cyclosporine. FIG. 4 illustrates that corneal staining improved (lowered) significantly following treatment, disappearing in 56% of patients. In particular, corneal staining, measured by the Oxford scale, significantly improved from a mean grade of 1.6±0.56 before cyclosporine 0.09% to 0.5±0.62 after treatment (P<0.000001, paired t-test). All eyes had at least grade 1 staining before treatment: 36 (56%) improved to grade 0 (absence of stain), 24 (38%) improved to grade 1, and 4 (6%) finished the study with grade 2 staining. Only 2 (3%) eyes showed no improvement in corneal staining, remaining at grade 2 after treatment.

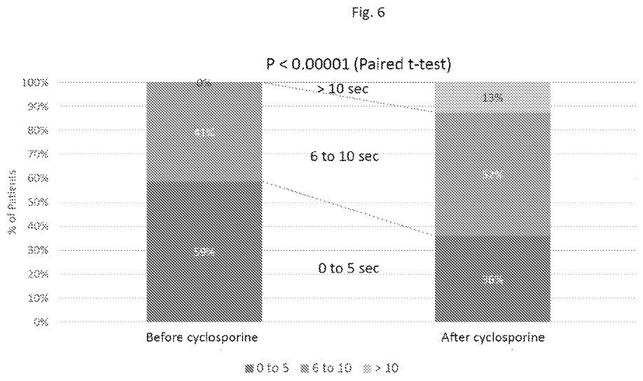

FIG. 6 illustrates the result of measurements of the tear breakup time (TBUT), measured before and after 28 days of treatment with 0.09% cyclosporine. As can be seen in FIG. 6, tear breakup time improved significantly following treatment. In particular, TBUT improved significantly from a mean of 5.2±2.2 seconds before treatment to 7.0±2.9 seconds after (P<0.000001, paired t-test). Mean improvement was 2.6±2.4 seconds. TBUT was 0 to 5 seconds in 37 (59%) and 23 (36%) eyes before and after cyclosporine 0.09% treatment, respectively (P<0.002, McNemar's chi-squared test), and 6 to 10 seconds in 26 (41%) and 33 (52%) patients before and after cyclosporine 0.09% treatment, respectively (P<0.03, McNemar's chi-squared test).

Conjunctival erythema, measured by the Schulze scale, also significantly improved with cyclosporine 0.09% treatment (mean score 19.1±8.9 and 15.3±6.7 before and after treatment, respectively; P<0.002, paired t-test). The lowest grading of 10 was observed in 23 (36%) eyes before and 36 (56%) after treatment (P<0.02, McNemar's chi-squared test). Other values for conjunctival redness are shown in Table 1.

TABLE 1

| Schulze Scale | # of eyes before | # of eyes after |
|---|---|---|
| Grading | treatment | treatment |
| Grade 10 | 23 (36%) | 36 (56%) |
| Grade 20 | 28 (44%) | 22 (34%) |
| Grade 30 | 10 (16%) | 6 (9%) |
| Grade 40 | 2 (3%) | 0 |
| Grade 50 | 1 (2%) | 0 |

64 patients completed the study. The absolute prediction error of 1-month spherical equivalent refractive outcome was 0.39±0.30 D vs 0.33±0.25 D (P<0.03) before and after treatment, respectively. The proportion of eyes that achieved the target refraction was greater based on measurements after topical cyclosporine 0.09% than would have occurred using pre-treatment measurements.

As can be seen from the results above, cataract surgery patients with dry eye who are prescribed topical cyclosporine 0.09% BID for 28 days pre-surgery showed a statistically significant improvement in the prediction error of the spherical equivalent outcome of surgery. Other measures of dry eye severity showed significant improvements after treatment.

The studies reported above enrolled patients with DED to assess the impact of a new formulation of cyclosporine on the refractive accuracy of cataract surgery. The present inventors found a statistically significant improvement in the prediction error of the spherical equivalent outcome of surgery when we used the measurements 28 days after cyclosporine 0.09% treatment had been initiated when compared to measurements performed before treatment. Other measures of dry eye severity, including corneal RMS HOAs, corneal staining, TBUT, conjunctival erythema, and SPEED scores, also showed significant improvements after cyclosporine 0.09% treatment.

Significant improvement was also noted for tear breakup time, giving evidence that treatment established a more stable tear film in dry eyes approaching surgery. This is particularly important for postoperative patients because achieving satisfaction with surgery depends not only on establishing clarity but also the ability to support prolonged reading and other visually intensive tasks.

Although many pharmacologic treatments for dry eye exist, cyclosporine 0.09% is the highest dose of cyclosporine currently approved by the FDA. It is also the only cyclosporine approved that includes nanomicellar technology for better penetration and drug absorption, as reported in Goldberg D F, Malhotra R P, Schechter B A, Justice A, Weiss S L, Sheppard J D. A Phase 3, Randomized, Double-Masked Study of OTX-101 Ophthalmic Solution 0.09% in the Treatment of Dry Eye Disease. Ophthalmology. September 2019; 126 (9): 1230-1237. doi:10.1016/j.ophtha.2019.03.050. Although there are some limits to the present disclosure, namely that the number of patients who initiated therapy and completed the study was lower than originally planned due to a high number of patients canceling surgery or wishing to withdraw to avoid noncrucial office visits, and that one patient withdrew because of intolerance to the study medication, a rate of discontinuation slightly better than the 2.4% discontinuation rate reported in the Goldberg US Food and Drug Administration phase 3 approval study of cyclosporine 0.09% noted above, the primary outcome measure—an improvement in refractive accuracy—showed a statistically significant result.

To the present inventors' knowledge, this is the first disclosure demonstrating improvements in refractive accuracy of cataract surgery with pretreatment cyclosporine 0.09%. The present results show that cyclosporine 0.09% has added utility beyond general dry eye treatment.

The present method may be combined with other treatments like artificial tears, warm compresses, dietary and habit modifications, and procedural remedies for DED.

The previous pivotal study (Goldberg, noted above) of cyclosporine 0.09% demonstrated superiority of this formulation over vehicle alone in treating DED. It is therefore envisioned that the improvements in refractive accuracy shown in the present disclosure, from cyclosporine 0.09%, would be similarly superior to those of its vehicle.

As with DED, corneal HOAs can be influenced by many factors, including seasonality, state of bodily hydration, hormonal changes, and any other factor that affects state of ocular hydration. Each of these can act at random and cause variance in the “smoothness” of the ocular surface. This may explain why 28% of cyclosporine 009%-treated patients had a worsening of their HOAs in this study. Despite these noteworthy and random variables, a significantly greater proportion of eyes, 44%, showed improvement in this metric.

It is interesting to note that, although all patients in the study had significant DED, the majority were symptom-free at baseline. SPEED scores were ≤10 in 75% and <5 in almost 40%. This corroborates earlier studies, including the PHACO study (Trattler W B, Majmudar P A, Donnenfeld E D, McDonald M B, Stonecipher K G, Goldberg D F, The Prospective Health Assessment of Cataract Patients' Ocular Surface (PHACO) study: the effect of dry eye; poster. Clin Ophthalmol. March 2017; 11:1423-1430. doi:10.2147/OPTH.S120159) that showed similar proportions of symptom-free patients, and it reinforces common clinic procedures to screen cataract surgery candidates for DED.

Even though certain specific embodiments are thoroughly described in the present application, it should be understood that the same concepts disclosed with respect to those specific embodiments are also applicable to other embodiments. Furthermore, individual elements of the formulations and methods disclosed herein are described with reference to particular embodiments only for the sake of convenience. It should be understood that individual elements of the formulations and methods disclosed herein are applicable to embodiments other than the specific embodiments in which they are described.

Furthermore, the contents of all documents discussed in the present disclosure are hereby incorporated by reference in their entirety.

In addition, it should be understood that the scope of the present disclosure is not limited to the above-described embodiments, and those skilled in the art will appreciate that various modifications and alterations are possible without departing from the scope of the present disclosure.

1. A method of preparing a subject for cataract surgery, the method comprising:
administering a solution comprising cyclosporine to an eye on which cataract surgery is to be performed.

2. The method of claim 1, wherein:
(i) the cyclosporine solution is topically administered;
(ii) the cyclosporine solution is administered twice daily;
(iii) the cyclosporine solution is administered for 28 days; or
(iv) any combination of (i)-(iii).

3. (canceled)

4. (canceled)

5. The method according to claim 1, wherein the solution comprising cyclosporine is an ophthalmic aqueous topical formulation consisting of about 0.087-0.093 wt % cyclosporine,
about 1.0 wt % hydrogenated 40 polyoxyl castor oil, about 0.05 wt % octoxynol-40,
about 0.3 wt % povidone,
about 0.05 wt % sodium chloride,
about 0.20-0.405 wt % sodium phosphate monobasic and
about 0.23-0.465 wt % sodium phosphate dibasic, adjusted to a pH of about 5 to about 8 with sodium hydroxide/hydrochloric acid, and
wherein the final volume is made up with water.

6. The method according to claim 5, wherein the cyclosporine is present in an amount of about 0.09 wt % of the formulation.

7. The method according to claim 5, wherein the pH of the formulation is about 6.6 to 7.0.

8. The method according to claim 1, wherein the solution comprising cyclosporine is an aqueous clear nanomicellar ophthalmic formulation comprising about 0.05-0.5% by weight cyclosporine, a polyalkoxylated alcohol and one or more polymers comprising HCO-40, HCO-60, HCO-80, HCO-100, polyoxyl 35 castor oil or combinations thereof.

9. (canceled)

10. (canceled)

11. (canceled)

12. (canceled)

13. The method according to claim 8, wherein said polymer comprises HCO-40 HCO-60, HCO-80, or combinations thereof and/or wherein said polymer is about 0.5-1.5% by weight of the formulation.

14. (canceled)

15. The method according to claim 8, wherein the polyalkoxylated alcohol comprises Octoxynol-40 and/or wherein the polyalkoxylated alcohol is about 0.02-4% by weight of the formulation.

16. (canceled)

17. The method according to claim 8, wherein the cyclosporine is about 0.05-0.2% by weight of the formulation.

18. The method according to claim 8, wherein said polymer comprises HCO-40, HCO-60, HCO-80, HCO-100 or combinations thereof; and wherein said polyalkoxylated alcohol is Octoxynol-40.

19. The method according to claim 8, wherein said polymer is about 0.5-1.5% by weight of the formulation; and said polyalkoxylated alcohol is Octoxynol-40 and is about 0.02-0.1% by weight of the formulation.

20. The method according to claim 8, wherein said polymer is about 0.5-1.5% by weight of the formulation; said polyalkoxylated alcohol comprises Octoxynol-40 and is about 0.02-0.1% by weight of the formulation; and the cyclosporine is about 0.05-0.2% by weight of the formulation.

21. A method of reducing ocular surface irregularity and/or reducing conjunctival erythema, the method comprising:
administering a solution comprising cyclosporine to an eye.

22. The method of claim 21, wherein;
(i) the cyclosporine solution is topically administered;
(ii) the cyclosporine solution is administered twice daily;
(iii) the cyclosporine solution is administered for 28 days; or
(iv) any combination of (i)-(iii).

23. (canceled)

24. (canceled)

25. The method according to claim 21, wherein the solution comprising cyclosporine is an ophthalmic aqueous topical formulation consisting of
about 0.087-0.093 wt % cyclosporine,
about 1.0 wt % hydrogenated 40 polyoxyl castor oil, about 0.05 wt % octoxynol-40,
about 0.3 wt % povidone,
about 0.05 wt % sodium chloride,
about 0.20-0.405 wt % sodium phosphate monobasic and
about 0.23-0.465 wt % sodium phosphate dibasic, adjusted to a pH of about 5 to about 8 with sodium hydroxide/hydrochloric acid, and
wherein the final volume is made up with water.

26. The method according to claim 25, wherein the cyclosporine is present in an amount of about 0.09 wt % of the formulation.

27. The method according to claim 25, wherein the pH of the formulation is about 6.6 to 7.0.

28. The method according to claim 21, wherein the solution comprising cyclosporine is an aqueous clear nanomicellar ophthalmic formulation comprising about 0.05-0.5% by weight cyclosporine, a polyalkoxylated alcohol and one or more polymers comprising HCO-40, HCO-60, HCO-80, HCO-100, polyoxyl 35 castor oil or combinations thereof.

29. (canceled)

30. (canceled)

31. (canceled)

32. (canceled)

33. The method according to claim 28, wherein:
said polymer comprises HCO-40 HCO-60, HCO-80, or combinations thereof and/or wherein said polymer is about 0.5-1.5% by weight of the formulation; and/or
wherein the polyalkoxylated alcohol comprises Octoxynol-40 and/or wherein the polyalkoxylated alcohol is about 0.02-4% by weight of the formulation.

34-60. (canceled)

61. A method for improving refractive accuracy and/or improving corneal staining, the method comprising administering an ophthalmic nanomicellar solution formulation comprising about 0.087 to about 0.093 wt % cyclosporine.

62. (canceled)
